(19) United States
(12) Patent Application Publication
(10) Pub. No.: US 2005/0119609 A1
(43) Pub. Date: Jun. 2, 2005

(54) Syringe mixer and syringe apparatus incorporating the mixer

(76) Inventor: Bruce S. McLean, Sandy, UT (US)

Correspondence Address: John M. Guynn;WORKMAN NYDEGGER, 1000 Eagle Gate Tower, 60 East South Temple, Salt Lake City, UT, 84111, US

(21) Appl. No.: 10/724,922

(22) Filed: Dec. 1, 2003

(51) Int. Cl. A61M 37/00

(52) U.S. Cl. 604/82

(57) ABSTRACT

A mixer enables a material to be transferred from a first syringe to a second syringe and enables another material to be transferred from the second syringe to the first syringe. The mixer includes a main body having a first surface, a second surface, first and second ends, first and second valve flaps, and is of a size and configuration adapted to be held at least partially within a tip of a syringe.

### BACKGROUND OF THE INVENTION

1. The Field of the Invention

This invention is in the field of methods and devices for mixing and dispensing compositions. More particularly, this application is directed to methods and devices for mixing and dispensing multi-part medical and dental compositions.

2. The Relevant Technology

Couplers have been employed in the art in order to couple one syringe in fluid communication with another syringe. Such couplers are typically configured to be positioned between the syringes by coupling one portion of the coupler to one syringe and another portion of the coupler to another syringe. Typical couplers have a passageway therethrough in order to enable material in one syringe to pass through the passageway of the coupler to the opposing syringe. After coupling the coupler between opposing syringes, material from one syringe may be delivered through the coupler into another syringe.

A variety of different uses for such so-called “syringe-to-syringe couplers” are available. For example, syringe-to-syringe couplers may be useful for connecting a large reservoir syringe to the small dose syringe so that the material stored in the large reservoir syringe may be transferred to a small dose syringe. Syringe-to-syringe couplers may also be used for back-filling a syringe or for combining materials in different syringes to form a mixture.

First and second syringes may also be coupled directly together without the use of a coupler therebetween in order to combine materials disposed within the syringes. The plungers of the syringes are alternatively compressed or actuated in cycles until the materials within the syringes mix.

There are many advantages to typical syringe-to-syringe mixing applications, both with respect to applications involving syringes which directly couple in order to combine material and with respect to syringes which couple through the use of a coupler. However, each of such typical syringe-to-syringe mixing applications feature a single, linear pathway which extends from one syringe to another or from one end of a coupler to an opposing end of the coupler.

Consequently, material in the distal tip portion of a first syringe is delivered in a substantially linear manner through the linear pathway to the distal tip portion of a second syringe. If the plunger of the second syringe is then pressed, substantially the same material delivered to the tip of the second syringe is delivered back along the same linear pathway in an opposite direction. As a result, substantially the same material originally delivered from the distal tip of the first syringe is returned back to the distal tip of the first syringe.

Thus, material located remotely from the tips can remain in such a remote position and fail to mix. Instead, substantially the same material is pushed back and forth along the linear pathway between the tips of the syringes. Overall, this phenomenon can result in inadequate mixing of the components from one syringe to another or can require long mixing times in order to mix the components.

In addition, existing couplers are often relatively complex and cumbersome. It would be an improvement to provide a simpler, but effective device for mixing the contents of coupled syringes.

### BRIEF SUMMARY OF THE PREFERRED EMBODIMENTS

In a syringe system for mixing a two-component composition, the present invention relates to a mixer for enabling mixing of the two components. The mixer is configured to be held at least partially within the tip of one of the syringes, where the syringes may be coupled together. The material held in a first syringe is introduced into a second syringe and then the mixture is repeatedly transferred back and forth between the two syringes. The mixer comprises: (i) a main body having a first surface, a second surface, a first end, and a second end; and (ii) a first valve flap and a second valve flap.

The first surface of the main body partially defines a first channel when held at least partially within the tip of a syringe. The first channel extends from the second end of the main body to the first end of the main body. The second surface of the main body partially defines a second channel when held at least partially within the tip of a syringe. The second channel extends from the first end of the main body to the second end of the main body.

In use, the mixer is held at least partially within the tip of a first syringe, which is coupled to a second syringe. The first and second syringes are coupled tip to tip. When mixing materials from the two syringes, a first flow path flows from the second syringe through the first channel to the first syringe. A second flow path flows from the first syringe through the second channel to the second syringe. Each channel includes a primary opening and a secondary opening. Material in each flow path enters the respective channel at the primary opening, exiting at the secondary opening.

In order to selectively open and close the first and second channels, a valve is located at the secondary opening of each channel. Each valve includes a valve seat and a valve flap. In one embodiment, the first valve seat is an inside surface of the barrel of the first syringe, and the second valve seat is an end surface of the tip of the first syringe. The first and second valve flaps extend outwardly from the main body, and are located at the first and second ends of the main body, respectively. Each valve flap may be a cantilevered flexible zone of the main body.

The first cantilevered flexible end can be configured to selectively close the secondary opening of the first channel when the end is positioned against the inside surface of the barrel of the first syringe. The second cantilevered flexible end can be configured to selectively close the secondary opening of the second channel when the end is positioned against the end surface of the tip of the first syringe.

In order to mix material in different syringes, the practitioner can deliver material back and forth between the syringes until the material is adequately mixed. Because of the configuration of the first and second channels, a substantially circular, asymmetric, non-linear flow pattern can be achieved when material is alternately delivered from a first syringe to a second syringe and vice versa. This allows convenient and efficient mixing of a first material in the first syringe and a second material in the second syringe. This is particularly useful for two-part type dental and medical compositions, such as epoxies, but is also useful in a variety of different applications in which mixing is desired.

In light of the bidirectional nature of the first and second channels, the material in the first syringe flows in a substantially different pathway than material flowing from the second syringe, and vice versa, creating the substantially circular flow pattern. Material expressed from each channel preferably exits outwardly into a side portion of a given syringe. This assists in circulating material from substantially different portions of the first and second syringes.

Also because of the side or outward exiting nature of the material, the flow path of material is asymmetric and turbulent. The asymmetric, turbulent flow enhances the mixing of the material. Since the flow pattern is asymmetric and substantially circular, the material is mixed in a quicker and more efficient manner.

In one embodiment, the mixer is substantially nonmovably affixed within the tip of a male syringe. According to one embodiment, the mixer may be integrally formed with the male syringe.

The mixer is configured to be held at least partially within the tip of one of the syringes at the juncture between the two syringes when the syringes are locked end to end directly to each other. A two syringe mixing apparatus of the present invention may thus comprise a first syringe, a second syringe, and a mixer configured to be held at least partially within the tip of the first syringe.

These and other advantages and features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by references to specific embodiments thereof, which are illustrated in the appended drawings. It is appreciated that these drawings depict only typical embodiments of the invention and are therefore not to be considered limiting of its scope. The invention will be described and explained with additional specificity and detail through the use of the accompanying drawings in which:

FIG. 1A is a perspective view of a mixer of the present invention;

FIG. 1B is a cross sectional view of a mixer of FIG. 1A held within the tip of a first syringe and the first syringe coupled to a second syringe;

FIG. 2 is an alternative cross sectional view of the mixer, first syringe, and second syringe of FIG. 1B;

FIG. 3 is a cross-sectional view of a barrel of a first syringe;

FIG. 4 is a cross-sectional view of a barrel of a second syringe;

FIG. 5A is a cross sectional view of a system of the present invention comprising the mixer of FIG. 1A and the syringe barrels of FIGS. 3 and 4. A material flows through a first flow path, while a second flow path is closed;

FIG. 5B is a view as in FIG. 5A in which the material flows through a second flow path while the first flow path is closed.

### DETAILED DESCRIPTION OF THE PREFERRED EMBODIMENTS

I. Introduction

As used herein, the term “syringe” and “syringes” includes syringes and other delivery means for delivering material, including, but not limited to, pumps, reservoirs, squeeze bottles, fluid bags, pressurized tanks, and other containers.

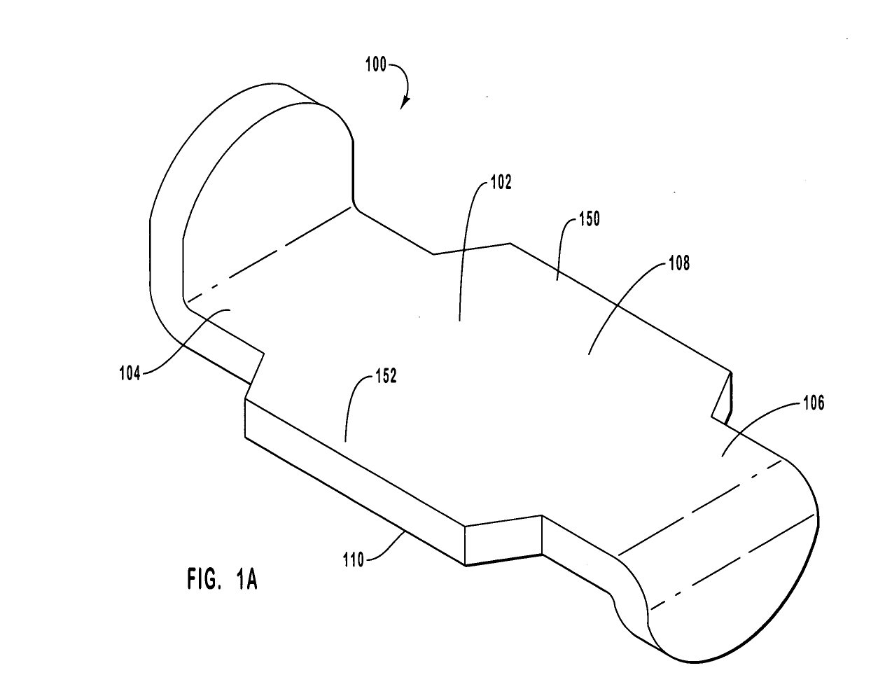

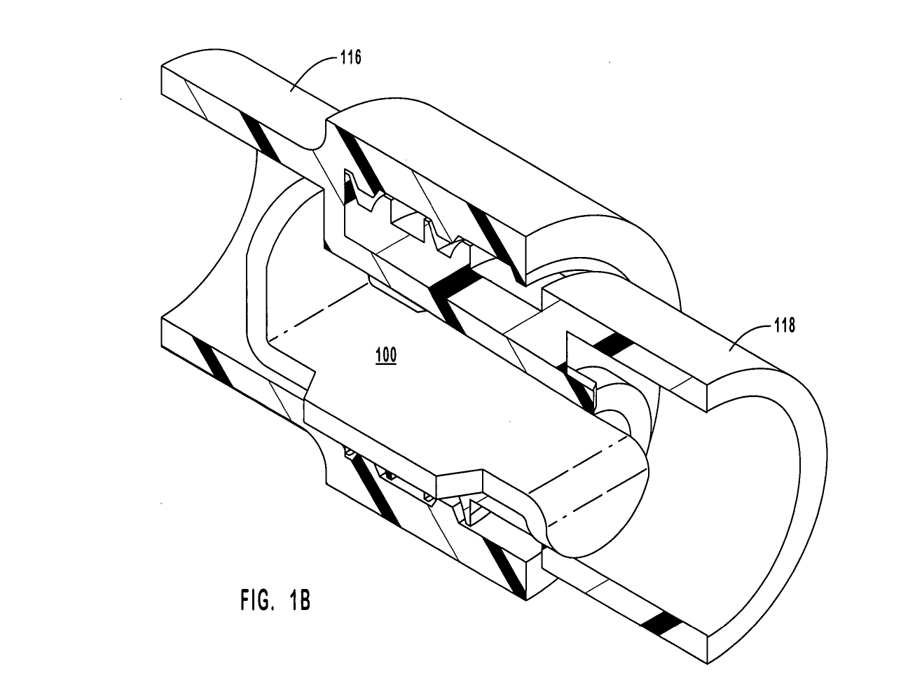

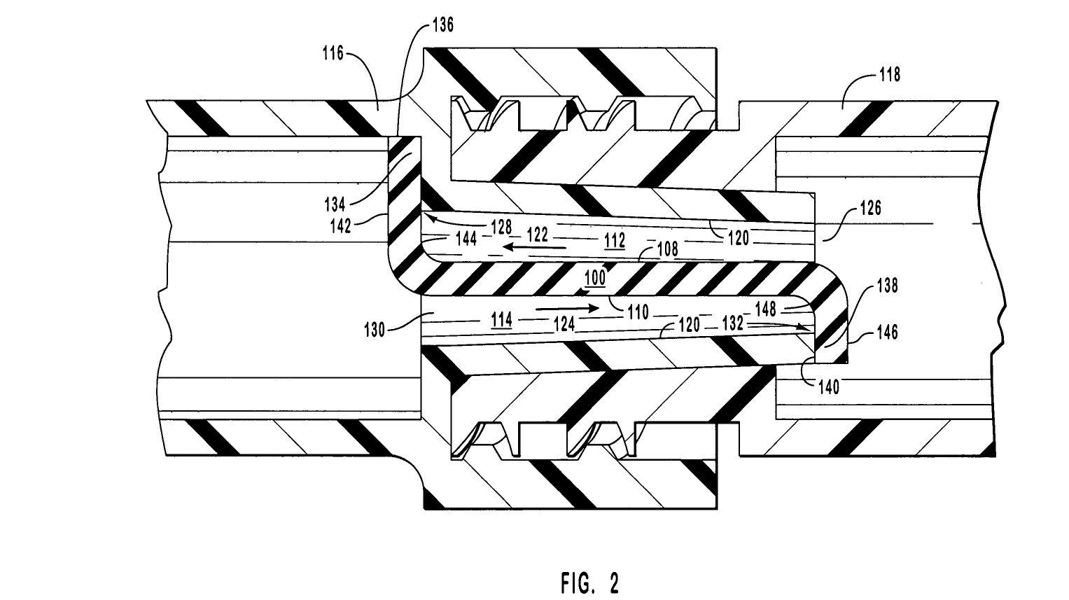

With reference now to FIGS. 1A, 1B and 2, an example of a mixer 100 of the present invention is shown. Mixer 100 is configured for enabling a material to be transferred from a first syringe for delivering material to a second syringe for delivering material and for enabling another material to be transferred from the second syringe to the first syringe.

II. An Exemplary Mixer

Mixer 100 has (i) a main body 102 having a first end 104, a second end 106, a first surface 108, and a second surface 110; and (ii) first and second valve flaps 134 and 138. When the mixer 100 is held at least partially within the tip of a syringe, the first surface 108 partially defines a first channel 112, while second surface 110 partially defines a second channel 114. First channel 112 extends from first end 104 of the main body 102 to second end 106 of main body 102. Second channel 114 extends from second end 106 of main body 102 to first end 104 of main body 102. First and second channels 112 and 114 are both closed in FIG. 2.

A. Main Body

In the mixer embodiment illustrated in FIGS. 1A-5B, the main body includes first and second surfaces 108 and 110 that partially define first and second channels 112 and 114 through the tip of first syringe 116. The mixer 100 may be integrally formed with the first syringe 116, or may be separately inserted so as to be frictionally held in place.

First channel 112 is defined by a first surface 108 of main body 102 and a first portion of interior surface 120 of the tip of the male first syringe 116. First channel 112 defines a first flow path 122. Also in this embodiment, second channel 114 is defined by an opposing second surface 110 of main body 102 and a second portion of interior surface 120 of the tip of the male first syringe 116. Second channel 114 defines a second flow path 124.

First channel 112 has a primary opening 126 at first end 104 of main body 102 and a secondary opening 128 (shown as being closed in FIG. 2) at second end 106 of main body 102. Second channel 114 has a primary opening 130 at second end 106 of main body 102 and a secondary opening 132 (shown as being closed in FIG. 2) at first end 104 of main body 102.

B. Valve Flaps

Mixer 100 includes a first valve flap 134 which operates in conjunction with first valve seat 136 to selectively close secondary opening 128 of first channel 112. A second valve flap 138 operates in conjunction with second valve seat 140 to selectively close secondary opening 132 of second channel 114. Valve seat 136 may comprise an interior surface of the barrel of first syringe 116, while valve seat 140 may comprise an end surface of the tip of first syringe 116.

Valve flap 134 has an exterior surface 142 and an interior surface 144. Valve flap 134 moves away from valve seat 136 when material is delivered from a first syringe within first channel 112 along first flow path 122 and against interior surface 144 of valve flap 134. Valve flap 134 thereby opens secondary opening 128 of first channel 112.

Valve flap 134 seals against valve seat 136 when material is delivered against an exterior surface 142 of valve flap 134, i.e., when material is delivered against exterior surface 142 of valve flap 134 from a second syringe. Valve flap 134 thereby closes secondary opening 128. Valve flap 134 preferably extends outwardly from main body 102, and is located at first end 104. As illustrated, valve flap 134 may be a cantilevered flexible zone of main body 102 that extends outwardly at about 90° (when in the closed configuration) relative to the central portion of main body 102.

Mixer 100 also includes a valve flap 138 which operates in conjunction with valve seat 140 to selectively close secondary opening 132 of second channel 114. In the illustrated embodiment, valve seat 140 comprises an end surface of the tip of first syringe 116.

Valve flap 138 has an exterior surface 146 and an interior surface 148. Valve flap 138 moves away from valve seat 140 when material is delivered from first syringe 116 within second channel 114 along second flow path 124 and against interior surface 148 of valve flap 138. Valve flap 138 thereby opens secondary opening 132 of second channel 114.

Valve flap 138 seals against valve seat 140 when material is delivered against an exterior surface 146 of valve flap 138, i.e., when material is delivered against exterior surface 146 of valve flap 138 from second syringe 118. Valve flap 138 thereby closes secondary opening 132. Valve flap 138 extends outwardly from main body 102, and is located at second end 106. As illustrated, valve flap 138 may be a cantilevered flexible zone of main body 102 that extends outwardly at about 90° (when in the closed configuration) relative to the central portion of main body 102. As in the illustrated embodiment, the valve flaps 134 and 138 may be integrally formed with the main body 102 as a single piece.

According to one embodiment, the mixer 100 also includes flanges 150 and 152. Flanges 150 and 152 extend from opposing sides of the central portion of main body 102 so as to hold mixer 100 within the tip of the male first syringe 116.

C. Materials

Mixer 100 may be comprised of a variety of different materials, although a material is preferred which is flexible enough that valve flaps 134, 138 can selectively open when pressure is applied on the interior surface thereof, yet close when pressure is applied on the exterior surfaces thereof. Examples of such materials which may be suitable in the present invention include polyethylene, polypropylene, neoprene, Santoprene, an olefin, such as J-VON, or another thermoplastic elastomer. The materials used in the mixer are preferably injection-molded. However, a variety of different materials and manufacturing methods can be employed, such as thermoset materials.

Materials such as polyethylene and polypropylene may be preferred where the materials to be mixed have a relatively low or moderate viscosity. When mixing higher viscosity materials, a harder material, for example acetal, having a durometer hardness of about 70, may be preferable. It is to be understood that the material chosen may be relatively flexible or more rigid, depending on the intended use.

III. An Exemplary System and Method of Use

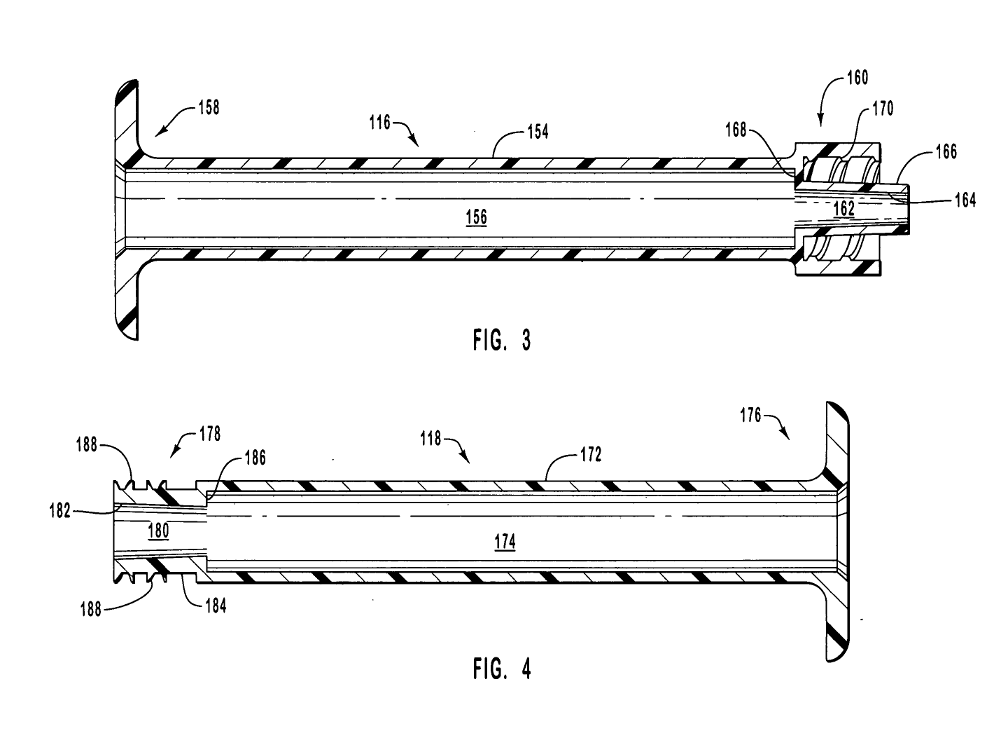

With reference now to FIGS. 3-5B, an example of a system of the present invention comprises (i) a first syringe 116 (FIG. 3); (ii) a second syringe 118 (FIG. 4); and (iii) mixer 100 (FIG. 1A) for enabling a practitioner to mix two components when first syringe 116 is coupled to second syringe 118.

In the embodiment of FIG. 3, first syringe 116 comprises a syringe barrel 154 and a syringe plunger (not shown in FIG. 3). Syringe barrel 154 has (i) a hollow main body 156 having a proximal end 158 and a distal end 160; and (ii) a hollow tip 162 coupled to and in fluid communication with main body 156.

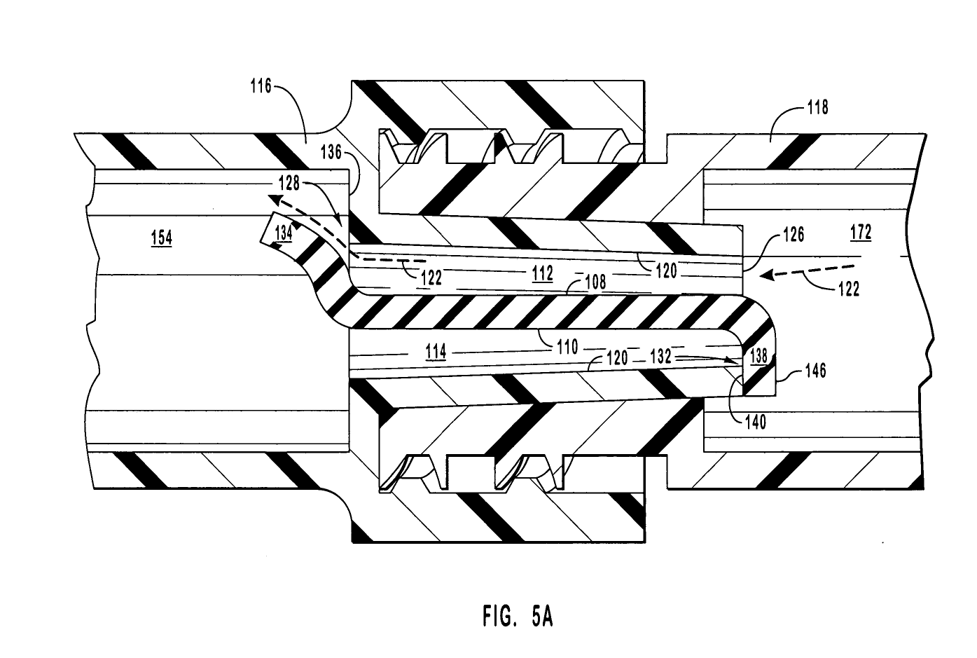

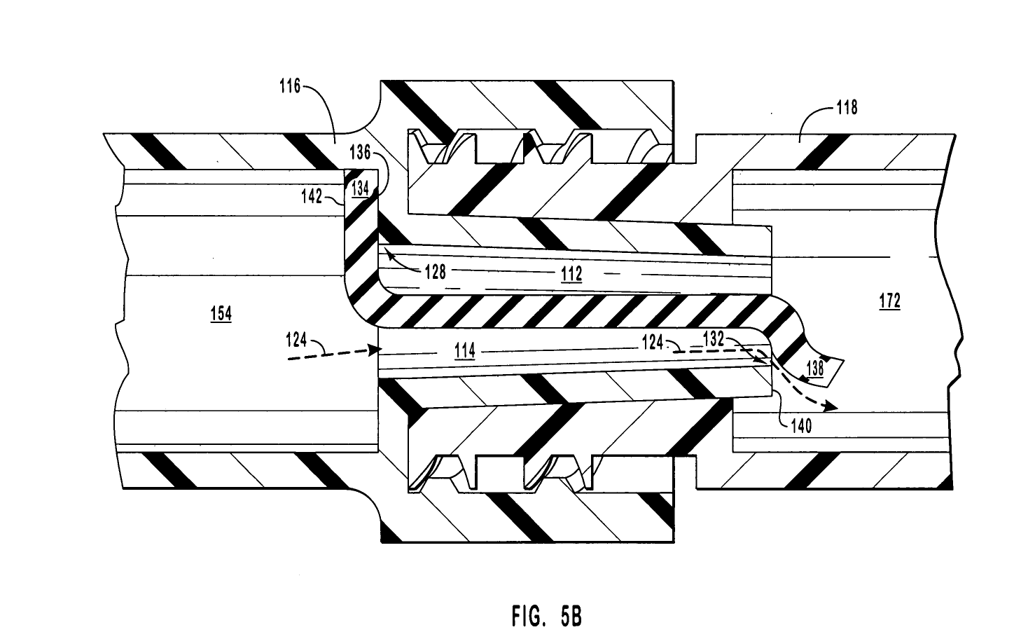

Tip 162 has an interior surface 164, an exterior surface 166, an internal circular shoulder 168, and female grooves 170 intended to mate with male threads of second syringe 118. As can be seen in FIG. 5A, upon extending first end 104 of mixer 100 past circular shoulder 168, first end 104 secures behind shoulder 168 and interior surface 164 of tip 162 frictionally engages flanges 150, 152 of mixer 100. In this manner, mixer 100 is held within the tip 162 of barrel 154.

In the embodiment of FIG. 4, second syringe 118 comprises a syringe barrel 172 and a syringe plunger (not shown in FIG. 4). Syringe barrel 172 has (i) a hollow main body 174 having a proximal end 176 and a distal end 178; and (ii) a hollow tip 180 coupled to and in fluid communication with main body 174.

Tip 180 has an interior surface 182, an exterior surface 184, an internal circular shoulder 186, and male threads 188 extending from exterior surface 184. Male threads 188 are configured to selectively mate directly with female grooves 170 of first syringe 116.

As can be seen in FIG. 5A, upon selectively coupling tip 180 of second syringe 118 to tip 162 of first syringe 116 after mixing apparatus 100 is placed within first syringe tip 162, the mixing apparatus 100 is frictionally held in place by flanges 150 and 152 contacting the interior surface 164 of first syringe tip 162. First barrel 154 and second barrel 172 are thus in fluid communication. Upon placing material and plungers within respective barrels 154 and 172, the material can be readily mixed by pressing against alternating plungers.

With continued reference now to FIG. 5A, first flow path 122 is shown. In operation, material delivered from second syringe 118 enters first channel 112 and travels along first flow path 122, exiting at secondary opening 128. As material is delivered along path 122, valve flap 134 is opened and unseated from valve seat 136. Flow path 122 extends out of secondary opening 128 past valve flap 134 in an outward manner, i.e., toward the wall of barrel 154. In the embodiment of FIG. 5A, material flows from second syringe 118. Thus, material from second syringe 118 pushes against exterior surface 146, causing second valve flap 138 to press against second valve seat 140, sealing secondary opening 132 of second channel 114.

With reference now to FIG. 5B, second flow path 124 is shown. In operation, material delivered from first syringe 116 enters second channel 114 and travels along second flow path 124, exiting at secondary opening 132. As material is delivered along path 124, valve flap 138 is opened and unseated from valve seat 140. Flow path 124 extends out of secondary opening 132 past valve flap 138 in an outward manner, i.e., toward the wall of barrel 172. In the embodiment of FIG. 5B, material flows from first syringe 116. Thus, material from first syringe 116 pushes against exterior surface 142, causing first valve flap 134 to press against first valve seat 136, sealing secondary opening 128 of first channel 112.

A schematic representation of the first and second flow paths 122 and 124 achieved when plungers in first and second barrels 154 and 172 are alternately compressed or actuated is shown in FIGS. 5A and 5B. Material flowing across mixer 100 is expressed through secondary openings 128 and 132 in an outward direction, i.e., toward the walls of respective barrels 154 and 172, aiding in circulation of material. Consequently, the exit pattern of path 122 is substantially remote from the entrance pattern of path 124, and vice versa, thereby providing for a circulating, mixing motion. Mixer 100 thus enables the circulation of material between syringe barrels in a substantially circular flow pattern, as opposed to movement of material back and forth between tips.

A variety of different materials may be mixed through the use of mixer 100, including liquids and powders and other compositions, such as A/B type compositions used in medicine and dentistry. Examples of A-B type materials which may be mixed with mixer 100 include epoxies, luting agents, powder-liquid combinations, powder-powder combinations, liquid-liquid combinations, two-part bleaching materials, and a variety of other materials known in the art or yet to be produced.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A mixer for use in a syringe system, the mixer comprising:
a main body having a first surface, a second surface, first and second ends, and of a size and configuration adapted to be held at least partially within a tip of a syringe;
the first surface partially defining a first channel when held at least partially within the tip of a syringe, the first channel extending from the second end of the main body to the first end of the main body,
the second surface partially defining a second channel when held at least partially within the tip of a syringe, the second channel extending from the first end of the main body to the second end of the main body;
a first valve flap extending outwardly from the main body and located at the first end of the main body for selectively opening and closing the first channel when the main body is held at least partially within the tip of a syringe; and
a second valve flap extending outwardly from the main body and located at the second end of the main body for selectively opening and closing the second channel when the main body is held at least partially within the tip of a syringe.

2. A mixer as recited in claim 1, wherein the main body further comprises at least one flange extending therefrom.

3. A mixer as recited in claim 2, wherein the first valve flap and the second valve flap are cantilevered flexible zones of the main body.

4. A mixer as recited in claim 1, wherein the main body and the first and second valve flaps are integrally formed as a single piece.

5. A mixer as recited in claim 1, the mixer being formed from at least one of a thermoplastic elastomer or a thermoset material.

6. A mixer as recited in claim 1, the mixer being formed from one or more of polyethylene, polypropylene, neoprene, or an olefin.

7. A mixer as recited in claim 1, the mixer being formed from a material having a durometer hardness of about 70.

8. A syringe and mixer system comprising:
a syringe having a tip; and
a mixer configured to be held at least partially within the tip of the syringe, the mixer comprising:
a main body having a first surface, a second surface, and first and second ends, and of a size and configuration adapted to be held at least partially within the tip of the syringe;
the first surface and a first portion of an interior surface of the tip of the syringe defining a first channel, the first channel extending from the second end of the main body to the first end of the main body, the first channel having a primary opening at the second end of the main body and a secondary opening at the first end of the main body;
the second surface and a second portion of an interior surface of the tip of the syringe defining a second channel, the second channel extending from the first end of the main body to the second end of the main body, the second channel having a primary opening at the first end of the main body and a secondary opening at the second end of the main body;
a first valve flap extending outwardly from the main body and located at the first end of the main body for selectively opening and closing the secondary opening of the first channel; and
is a second valve flap extending outwardly from the main body and located at the second end of the main body for selectively opening and closing the secondary opening of the second channel.

9. A syringe and mixer system as recited in claim 8, wherein the syringe is a male syringe.

10. A syringe and mixer system as recited in claim 9, wherein the syringe and mixer are integrally formed together.

11. A two syringe mixing apparatus, comprising:
a first syringe having a tip;
a second syringe having a tip; and
a mixer configured to be held at least partially within the tip of the first syringe, the mixer comprising:
a main body having a first surface, a second surface, and first and second ends, and of a size and configuration adapted to be held at least partially within the tip of the first syringe;
the first surface and a first portion of an interior surface of the tip of the first syringe defining a first channel, the first channel extending from the second end of the main body to the first end of the main body, the first channel having a primary opening at the second end of the main body and a secondary opening at the first end of the main body;
the second surface and a second portion of an interior surface of the tip of the first syringe defining a second channel, the second channel extending from the first end of the main body to the second end of the main body, the second channel having a primary opening at the first end of the main body and a secondary opening at the second end of the main body;
a first valve flap extending outwardly from the main body and located at the first end of the main body for selectively opening and closing the secondary opening of the first channel;
a second valve flap extending outwardly from the main body and located at the second end of the main body for selectively opening and closing the secondary opening of the second channel; and
the second syringe being coupled to the first syringe.

12. A two syringe mixing apparatus as recited in claim 11, wherein the apparatus is configured such that material flowing through the channels is expressed through the secondary openings in an outward direction such that the exit pattern of a flow path of one material is substantially remote and nonparallel with the entrance pattern of a flow path of another material, thereby creating a churning action to optimally mix the materials.

13. A two syringe mixing apparatus as recited in claim 11, wherein a flow path defined by the first channel is substantially distinct from a flow path defined by the second channel.
